(19) United States
(12) Patent Application Publication
(10) Pub. No.: US 2023/0071672 A1
(43) Pub. Date: Mar. 9, 2023

(54) PRESERVING FLUOROPHORE FUNCTION

(71) Applicant: Lions VisionGift, Portland, OR (US)

(72) Inventor: Khoa D. Tran, Beaverton, OR (US)

(21) Appl. No.: 17/929,579

(22) Filed: Sep. 2, 2022

Related U.S. Application Data
(60) Provisional application No. 63/241,912, filed on Sep. 8, 2021.

(51) Int. Cl. G01N 33/533

(52) U.S. Cl. CPC G01N 33/533

(57) ABSTRACT

Fluorophore compositions are described herein in which the functionality of fluorophores contained therein are preserved during extended storage. Methods of making and using such compositions are also described herein.

### CROSS-REFERENCE TO RELATED APPLICATIONS

This application claims priority to U.S. Provisional Patent Application No. 63/241,912, entitled “PRESERVING FLUOROPHORE FUNCTION”, filed on Sep. 8, 2021, which is hereby incorporated by reference herein in its entirety.

### FIELD OF THE INVENTION

The present disclosure relates generally to compositions and associated methods for use in fluorescent staining of cells, and more particularly for preserving the functionality of fluorophores in such compositions.

### BACKGROUND

The evaluation of cell viability via selective staining techniques continues to be an important tool for assessing cell populations in samples and culture. Current approaches can involve the use of a single dye or a combination of dyes—often fluorescent dyes—where each exhibits differential staining of cells based on cell membrane integrity or intracellular activity. Calcein AM, propidium iodide (PI), 4′,6-diamidino-2-phenylindole (DAPI), and Hoechst stains are common fluorescent dyes used in cell biology research. These dyes often cannot be stored frozen without significant loss of function; rather, they must be mixed fresh prior to each use. For example, calcein AM solutions have been observed to lose functionality if stored over night at 4° C., or frozen and thawed. Thus, once an amount of the dye solution is made, it must either be used or discarded.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIGS. 1A through 1C show photomicrographs of three corneas under illumination by a fluorescence microscopy light source after staining with a calcein AM/sodium hyaluronate mixture immediately after preparation (FIG. 1A), after frozen storage for five days (FIG. 1B), and three months (FIG. 1C).

FIGS. 2A through 2F show photomicrographs of corneas under illumination by a fluorescence microscopy light source after staining with calcein AM immediately after preparation (FIG. 2A) or after being stored under 4° C. refrigeration overnight (FIG. 2B); for five days (FIG. 2C); for one month (FIG. 2D); or for three months (FIG. 2E). FIG. 2F shows a cornea stained with a mixture of calcein AM and sodium hyaluronic acid that had been stored frozen for 12 months.

FIGS. 3A through 3F show photomicrographs of a population of corneal cells stained with a mixture of calcein AM, PI, and Hoechst with sodium hyaluronic acid that was stored frozen for 12 months. FIG. 3A shows the calcein AM fluorescent signal, FIG. 3B the PI fluorescent signal, and FIG. 3C the Hoechst fluorescent signal. Combined signals are shown for calcein AM and PI in FIG. 3D, calcein AM and Hoechst in FIG. 3E, and calcein AM, PI, and Hoechst in FIG. 3F.

FIGS. 4A through 4D show photomicrographs of a population of corneal cells stained with a mixture of calcein AM, PI, and Hoechst with sodium hyaluronic acid that was stored frozen for 32 months. FIG. 4A shows the calcein AM fluorescent signal, FIG. 4B the PI fluorescent signal, and FIG. 4C the Hoechst fluorescent signal. Combined signals are shown for calcein AM, PI, and Hoechst in FIG. 4D.

### DETAILED DESCRIPTION

The components of the embodiments as generally described and illustrated herein can be arranged and designed in a wide variety of different configurations. Thus, the following more detailed description of various embodiments is not intended to limit the scope of the present disclosure, but is merely representative of various embodiments. It will be appreciated that various features are sometimes grouped together in a single embodiment, figure, or description thereof for the purpose of streamlining the disclosure. Many of these features may be used alone and/or in combination with one another.

As used herein, the term “fluorescent” refers to the property of a molecule whereby, upon irradiation with light of a given wavelength or wavelengths, the molecule becomes excited and emits light of a longer wavelength or wavelengths. The term “fluorophore” as used herein refers to a fluorescent molecule.

The present disclosure describes fluorophore compositions in which the staining and fluorescence activity of fluorophores provided therein are substantially preserved during storage and even freezing. The inventors have surprisingly found that fluorescent dye can be combined with hyaluronic acid to confer such storage stability to fluorophores in the dye. In accordance therewith, a fluorophore composition can comprise a dye preparation combined with a hyaluronic acid salt, where the dye preparation comprises one or more types of fluorophore. In some embodiments, the dye preparation can comprise one to five types of fluorophore or fluorophore precursor.

In various embodiments, the dye preparation includes a fluorophore precursor that can be converted into a fluorophore at some time after formulation of the dye preparation. In one non-limiting example, the dye may include calcein AM. Calcein AM is a non-fluorescent acetomethoxy-derivate of calcein which can be transported through the cellular membrane into live cells. Esterases in the intracellular environment remove the acetomethoxy group, trapping the now-green-fluorescent molecule inside the cell. Other calcein precursors include, without limitation, calcein blue AM and calcein red-orange AM. Therefore, it should be noted that references in the present disclosure to fluorophores or types of fluorophore provided by a fluorophore composition also encompass fluorophores that are initially incorporated into the composition as precursors.

The one or more types of fluorophore may include one or more fluorescent dyes including, but not limited to, fluorescein isothiocyanate (FITC), fluorescein amidite (FAM), erythrosine, Rose Bengal, Methylene Blue, laser dyes, rhodamine dyes (including Rhodamine 6G, Rhodamine B, Rhodamine 123, Sulforhodamine 101, Sulforhodamine B), anthraquinone dyes, Auramine O, Texas Red, ATTO dyes, Acridine orange, Acridine yellow, Alexa Fluor dyes, 7-aminoactinomycin D, 8-anilinonaphthalene-1-sulfonate, benzanthrone, 5,12-bis(phenylethynyl)naphthacene, 9,10-bis(phenylethynyl)anthracene, calcein, calcein blue, calcein red orange, carboxyfluorescein, carboxyfluorescein diacetate succinimidyl ester, carboxyfluorescein succinimidyl ester, 1-chloro-9,10-bis(phenylethynyl)anthracene, 2-chloro-9,10-bis(phenylethynyl)anthracene, 2-chloro-9,10-diphenylanthracene, coumarin, cyanine dyes (including Cy3, Cy3.5, Cy3B, Cy5, Cy5.5, cy7), DiOC6, SYBR Green I, DAPI, DRAQ 5, DyLight Fluor, Fluo-4, FluoProbes, carboxyfluorescein diacetate succinimidyl ester, eosin, Eosin B, Eosin Y, Fluorescein, fluorescein isothiocyanate, fluorescein amidite, Indian Yellow, merbromin, Fluoro-Jade stain, Fura-2, Fura-2-acetoxymethyl ester, Green fluorescent protein, Hoechst stain, Indo-1, Lucifer yellow, luciferin, merocyanine, oxazin dyes, Cresyl Violet, Nile Blue, Nile Red, Oil Red O, perylene, phenanthridine dyes, ethidium bromide, propidium iodide, phloxine, phycobilin, phycoerythrin, phycoerythrobilin, pyranine, RiboGreen, RoGFP, rubrene, SYBR Green I, Synapto-pHluorin, tetraphenyl butadiene, tetrasodium tris(bathophenanthroline disulfonate)ruthenium(II), toluidine blue O, TSQ, umbelliferone, or Yellow fluorescent protein.

Particular dyes may be selected singly or in combination based on their binding activity or other mode of action. In some embodiments, the dye preparation includes a cell viability dye, which herein refers to a dye that can be used singly or in combination with other dyes to distinguish between living and dead cells in a population. For example, one type of cell viability dye may selectively bind intracellular material while being impermeant to living cell membranes and thereby selectively stain dead or dying cells. Another type of cell viability dye may permeate living cell membranes and selectively bind a product of active intracellular processes, and thereby selectively stain living cells. Cell viability dyes include without limitation propidium iodide (PI), hexidium iodide, carbocyanine, rhodamine 123, tetra methyl rhodamine, dialkylaminophenylpolyenylpyridinium, aminonaphthylethenylpyridinium, resazurin, formazan, red-fluorescent ethidium homodimer-1, calcein, Hoechst stain, tetrasodium (6E,6′E)-6,6-[(3,3′-dimethylbiphenyl-4,4′-diyl)di(1E)hydrazin-2-yl-1-ylidene]bis(4-amino-5-oxo-5,6-dihydronaphthalene-1,3-di sulfonate) (Evans blue), (3Z,3′Z)-3,3′-[(3,3′-dimethylbiphenyl-4,4′-diyl)di(1Z)hydrazin-2-yl-1-ylidene]bis(5-amino-4-oxo-3,4-dihydronaphthalene-2,7-disulfonic acid) (Trypan blue), 7-aminoactinomycin D (7-AAD). In some embodiments, the dye preparation includes a nucleic acid dye.

The dye preparation may include one or more types of fluorophore having particular excitation and emission spectra. In some embodiments, the dye preparation can include a fluorophore having an emission maximum located at a wavelength selected from about 400 nm to about 500 nm, about 500 nm to about 600 nm, about 525 nm to about 575 nm, about 550 nm to about 650 nm, about 650 nm to about 750 nm, and about 750 nm to about 850 nm.

In some embodiments, the dye preparation includes at least one of calcein AM, Hoechst 33342, Hoechst 33258, propidium iodide, or DAPI.

In some embodiments, the dye preparation can include at least one fluorophore or fluorophore precursor that is conjugated to another molecule having significance or specificity with regard to a target cell, such as an enzyme, antibody, or other fluorophore. In particular, fluorophores are used in conjugation with antibodies as detection reagents in a number of cell biology applications. In an embodiment, the dye preparation includes a fluorophore or fluorophore precursor conjugated to an antibody.

The dye preparation can vary in composition, as it depends on the particular fluorophore or combination of fluorophores included therein to meet the needs of applications of interest. In various embodiments, the amount of dye preparation in the fluorophore composition includes about 1 μg to about 10 μg of fluorophore or fluorophore precursor per milliliter of the fluorophore composition. In some embodiments, the fluorophore or fluorophore precursor is present in the fluorophore composition at a concentration of about 5 nM to about 20 nM.

The dye preparation may comprise a solvent or carrier selected to facilitate incorporation of the fluorophores into the dye preparation or the combination of the preparation with hyaluronic acid as described in further detail below. For example, dimethyl sulfoxide (DMSO) is often used as a solvent in the preparation of fluorescent dyes for use. In a particular example, calcein AM—a solid that is only slightly soluble in water and alcohol—is often dissolved in DMSO and diluted with a balanced salt solution for use in staining cells. Accordingly, the use of DMSO to make dye preparations for use in fluorophore compositions is also encompassed by the present disclosure. In various embodiments, the amount of DMSO used may be limited to the minimum amount effective to incorporate the fluorophore(s) into the dye preparation. In some embodiments, DMSO constitutes less than about 1 wt % of the fluorophore composition. In an embodiment, the fluorophore composition is substantially free of DMSO.

As noted above, a fluorophore composition in accordance with the present disclosure can comprise a combination of the above-described dye preparation with a hyaluronic acid salt. In particular, a hyaluronic acid salt may be selected that is suited for combination with a particular dye preparation to form a stable composition, and more particularly a salt that is suited for constituting a solution configured to provide a salt balance and/or pH that is compatible with the cell sample or cell culture to be stained. In particular embodiments, the hyaluronic acid salt is sodium hyaluronate. However, it will be understood that hyaluronic acid salts encompassed by the present disclosure are not necessarily limited by the foregoing.

The amount of hyaluronic acid salt may be selected in part to provide a desired preservative effect for the particular fluorophore(s) under anticipated storage conditions. The amount of hyaluronic acid salt may also be selected in part to confer particular characteristics e.g., osmolarity, pH, to the fluorophore composition. This may be achieved in combination with other ingredients known to be useful in formulating balanced salt solutions, including without limitation: inorganic salts, such as sodium chloride; buffering agents including monobasic sodium phosphate, dibasic sodium phosphate; acids such as hydrochloric acid; and bases such as sodium hydroxide, amino acids, vitamins, inorganic salts, glucose, and the like. In some embodiments, the amount of hyaluronic acid salt is about 0.5 wt % to about 3 wt %.

An aspect of the present disclosure is that fluorophore compositions comprising hyaluronic acid can provide more effective staining of cells and tissues. In some embodiments, combining a hyaluronic acid salt with a dye preparation can provide a fluorophore composition in which fluorophores are more homogeneously incorporated. As a result, such a fluorophore composition may stain a sample more consistently and evenly than a composition including the same fluorophores but lacking hyaluronic acid. In a particular aspect of the above, it is contemplated that fluorophore compositions comprising hyaluronic acid can be beneficial for staining and imaging preparations of living cells. For example, it has been found that hyaluronic acid gel is useful as a medium for mounting living corneal tissue for imaging purposes. Accordingly, in some embodiments, the fluorophore composition may be formulated for compatibility with mounting and imaging a living tissue preparation, such as excised corneal tissue.

An aspect of the fluorophore compositions described herein is that, unlike conventional fluorescent dye formulations, they can be stored before or between uses without significant loss of functionality. Of particular significance is the preservation of fluorescence i.e., the capacity for excitation by a light stimulus and the resultant emission of light in levels sufficient for identification of the fluorophore's location. One parameter of fluorescence effectiveness is the fluorophore's ability to absorb light as reflected in its molar absorption coefficient, which refers to the amount of light of a given wavelength absorbed per mole of the fluorophore over a known linear path length. Another parameter is quantum yield, which refers to the ratio of photons absorbed to photons emitted through fluorescence. In some embodiments, a functionality of the one or more types of fluorophore in the fluorophore composition is substantially unchanged by storage of the composition. In some embodiments, the functionality so preserved can be molar absorption coefficient, quantum yield, or a combination of these. In some embodiments, the functionality preserved is the labeling efficiency of one or more fluorophore types. In view of this aspect, making a fluorophore composition as described herein using a fluorophore of interest can provide a way to preserve a functionality of said fluorophore against degradation arising from storage.

In accordance with the above, fluorophore compositions described herein maintain the functionality of fluorophores therein under various storage regimes over a wide range of temperatures, including refrigeration and freezing. In some embodiments, such storage includes temperatures from about −80° C. to about 4° C. For example, the compositions can be stored under refrigeration or other storage providing temperatures from about 0° C. to about 4° C. The compositions can be stored frozen, for example in an ultra-low temperature freezer or other environment providing temperatures from about −80° C. to about 0° C., or from about −80° C. to about −20° C., or from about −80° C. to about −40° C.

It is contemplated that the fluorophore compositions may be stored at any of these storage temperatures for an indefinite period of time without substantial loss of fluorophore functionality. In some embodiments, the fluorophore composition may be stored at a storage temperature for about one day to about three years, or about one year to about three years, or about one year to about two years, or about two years to about three years. The fluorophore compositions can also be removed from frozen storage, thawed for use, and refrozen for further storage. Accordingly, the storage regime may include a number of freeze-thaw cycles, for example two or more freeze-thaw cycles.

Fluorophore compositions described herein may be made by combining an amount of a dye preparation with an amount of hyaluronic acid. In some embodiments, the hyaluronic acid is provided as a salt in solution. The solution may further comprise ingredients suitable for the particular application. For example, if the composition is to be used to stain live cells, the solution may further include inorganic salts, buffers, acids, bases, amino acids, vitamins, inorganic salts, glucose, and the like. In a non-limiting example, the hyaluronic acid salt solution may comprise an aqueous solution of sodium hyaluronate, anhydrous dibasic sodium phosphate, monobasic sodium phosphate, sodium chloride, with an amount of hydrochloric acid and/or sodium hydroxide added to adjust pH. One such hyaluronic acid salt solution is available as Provisc® (Alcon), where each ml contains: 10.0 mg sodium hyaluronate; 0.56 mg dibasic sodium phosphate, anhydrous; 0.04 mg monobasic sodium phosphate; 8.4 mg sodium chloride; hydrochloric acid and/or sodium hydroxide; and water. In some embodiments, hyaluronic acid salt may be incorporated into cell culture medium to provide the hyaluronic acid salt solution.

A method of making a fluorophore composition can comprise preparing a dye preparation using one or more types of fluorophore or fluorophore precursor, and combining the dye preparation with a hyaluronic acid salt solution. Preparing the dye preparation may comprise combining the fluorophore(s) with a solvent or carrier selected to facilitate incorporation of the fluorophore(s) into the dye preparation and/or combining the dye preparation with hyaluronic acid. As discussed above, DMSO is one example of such a solvent or carrier. Accordingly, in an embodiment the dye preparation may be made by dissolving one or more fluorophore types or fluorophore precursors in DMSO. Where two or more fluorophores are used, they may each be combined with the solvent or carrier in one step or in separate steps.

One approach may comprise preparing an amount of the dye preparation; preparing a hyaluronic acid salt solution; and then combining said amount of the dye preparation with an amount of hyaluronic acid salt solution so as to dilute the one or more fluorophore types to a desired concentration. The hyaluronic acid salt solution may also be formulated so as provide a desired concentration of hyaluronic acid in the final composition. In some embodiments, an amount of dye preparation is combined with an amount of hyaluronic acid salt solution so as to provide about 1 μg to about 10 μg of fluorophore or fluorophore precursor per milliliter of the fluorophore composition. In some embodiments, the amount of dye preparation provides fluorophore or fluorophore precursor in the fluorophore composition at a concentration of about 5 nM to about 20 nM. In some embodiments, the hyaluronic acid salt solution comprises an amount of hyaluronic acid salt corresponding to about 0.5 wt % to about 3.0 wt % of the fluorophore composition.

It may be useful—and more efficient—to prepare an amount of fluorophore composition sufficient for multiple uses. Such an approach can be additionally useful when the fluorophore composition is to be frozen for storage, as the smaller volume of a single-use aliquot can be more readily thawed for use than a bulk amount. Furthermore, this can reduce the number of freeze-thaw cycles to which any portion of the composition is subjected, further enhancing the functional storage life of the composition. Accordingly, a method of making the composition can further comprise dividing the fluorophore composition into a number of single-use aliquots.

Fluorophore compositions described herein are contemplated for use in a number of cell biology analysis techniques, such as in-vitro staining of one or more cells for subsequent imaging. In accordance with the present disclosure, a method of preparing a cell for imaging can comprise contacting the cell with an amount of a fluorophore composition as described herein. In some embodiments, the amount of fluorophore composition may be a single-use aliquot as discussed above. The amount of composition used may have been previously stored at a storage temperature in a range of temperatures described above. Accordingly, before applying the composition to the cell, a preliminary step can comprise raising the temperature of the amount of composition from the storage temperature to a temperature appropriate for use, for example a temperature at or near the cell culture temperature.

### EXAMPLES

### Example 1—Calcein AM Activity is Preserved by Sodium Hyaluronic Acid

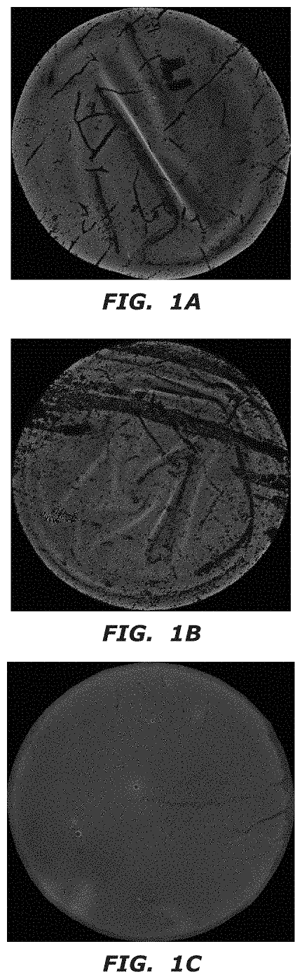

Calcein AM (2.5 μg/mL) was prepared with 1.7% sodium hyaluronic acid in a balanced salt solution. Three corneas were stained for 15 minutes and imaged under the same conditions. Imaging of the corneas under illumination by a fluorescence microscopy light source is shown in FIGS. 1A-1C. The cornea shown in FIG. 1A was stained with the mixture immediately after preparation. The cornea shown in FIG. 1B was stained with the mixture after the mixture had been stored frozen for 5 days. The cornea shown in FIG. 1C was stained with the mixture after the mixture had been stored frozen for 3 months. As shown each cornea the mixture stained living cells and yielded a clear green fluorescence signal.

### Example 2—Calcein AM Activity is Maintained after Long-Term Frozen Storage in Sodium Hyaluronic Acid

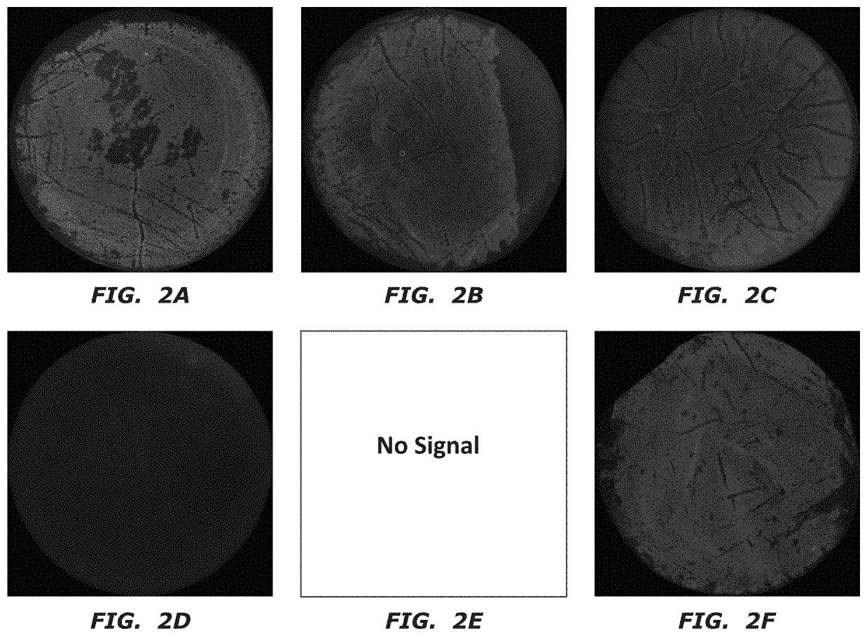

Calcein-AM (2.5 μg/mL) was prepared according to manufacturer's recommendations. All corneas were stained for 15 minutes and imaged under the same conditions. Imaging of the corneas under illumination by a fluorescence microscopy light source (using the same imaging parameters) is shown in FIGS. 2A-2F, where FIG. 2A shows a cornea stained with freshly prepared calcein AM, and FIGS. 2B-2E show corneas stained with the calcein AM stored under 4° C. refrigeration and protected from light for the following periods of time:

FIG. 2B: overnight;

FIG. 2C: five days;

FIG. 2D: one month;

FIG. 2E: three months.

As can be seen, the fluorescence signal produced by each stain loses intensity with the storage time length, and is completely lost by three months' storage time. In contrast, a cornea stained with a mixture of calcein AM (2.5 μg/mL) and 1.7% sodium hyaluronic acid stored frozen for 12 months shows a strong fluorescence signal (FIG. 2F).

### Example 3—Sodium Hyaluronic Acid Preserves Function of Nuclear Stains Propidium-Iodide (PI) and Hoechst During Long Term Frozen Storage

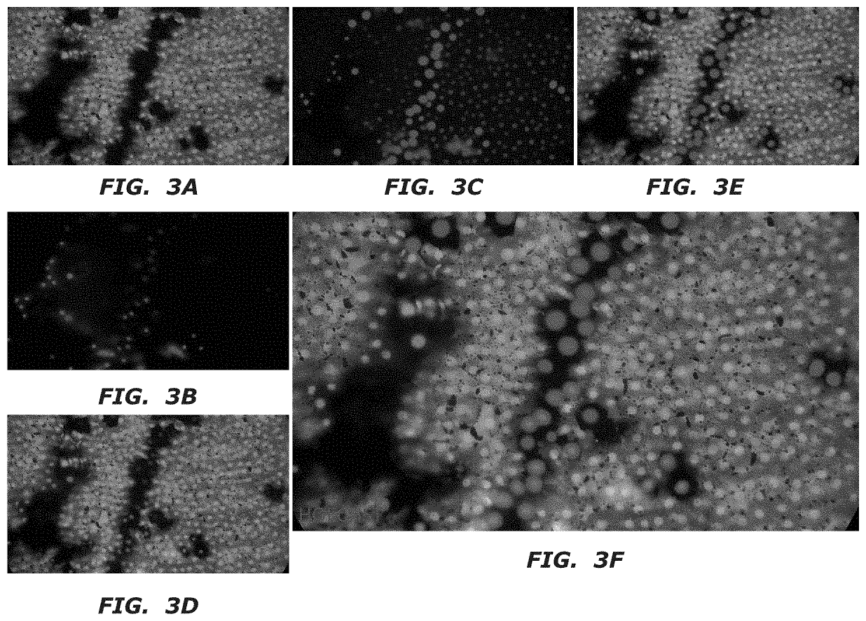

A mixture of calcein AM, PI, and Hoechst was prepared in sodium hyaluronic acid and stored frozen for 12 months. FIGS. 3A-3F show various images of one population of corneal cells stained with the mixture after thawing. Calcein AM signal (see FIG. 3A) indicates live cells while PI signal (see FIG. 3B) indicates dead cells (cells with compromised membranes). Hoechst (signal shown in FIG. 3C) is a nuclear marker that can stain both live and dead cells. Combined signals for calcein AM and PI are shown in FIG. 3D, calcein AM and Hoechst in FIG. 3E, and calcein AM, PI, and Hoechst in FIG. 3F. As shown, the functionality of each fluorophore type was preserved during storage and the composition remained effective for assessing cell viability.

### Example 4—Sodium Hyaluronic Acid Preserves Function of Nuclear Stains Propidium-Iodide (PI) and Hoechst During Extended Period of Frozen Storage

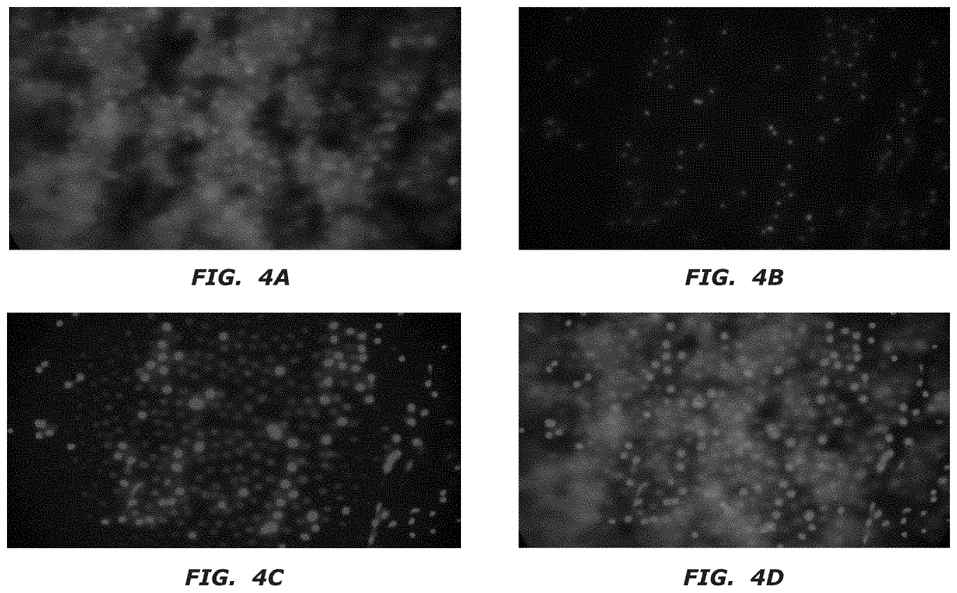

Corneal cells were stained with a mixture of calcein AM, PI, and Hoechst in sodium hyaluronic acid that had been stored frozen for 32 months. FIG. 4A shows the calcein AM fluorescent signal, FIG. 4B the PI fluorescent signal, and FIG. 4C the Hoechst fluorescent signal. Combined signals are shown for calcein AM, PI, and Hoechst in FIG. 4D. As shown, the functionality of each fluorophore type was preserved during an extended storage period and the composition remained effective for assessing cell viability.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “substantially” and “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. All ranges also include both endpoints.

It will be apparent to those having skill in the art that many changes may be made to the details of the above-described embodiments without departing from the underlying principles of the invention. The scope of the present invention should, therefore, be determined only by the following claims.

1. A fluorophore composition, comprising an amount of a dye preparation and an amount of a hyaluronic acid salt, wherein the dye preparation comprises one or more types of fluorophore or fluorophore precursor.

2. (canceled)

3. The fluorophore composition of claim 1, wherein the dye preparation includes at least one of a nucleic acid dye and a cell viability dye.

4. (canceled)

5. The fluorophore composition of claim 1, wherein the dye preparation comprises at least one of calcein AM, Hoechst 33342, Hoechst 33258, propidium iodide, or DAPI.

6. (canceled)

7. The fluorophore composition of claim 1, wherein the fluorophore or fluorophore precursor is conjugated to an antibody.

8. (canceled)

9. The fluorophore composition of claim 1, wherein the amount of the dye preparation includes about 1 μg to about 10 μg of fluorophore or fluorophore precursor per milliliter of the fluorophore composition.

10. The fluorophore composition of claim 1, comprising less than about 1 wt % DMSO.

11. (canceled)

12. The fluorophore composition of claim 1, wherein the amount of hyaluronic acid salt is about 0.5 wt % to about 3 wt %.

13. The fluorophore composition of claim 1, wherein a functionality of the one or more types of fluorophore is substantially unchanged by storage of the fluorophore composition, wherein the storage is at a temperature of from about −80° C. to about 4° C. for a storage period of about one day to about three years.

14. The fluorophore composition of claim 13, wherein the functionality comprises one or more of labeling efficiency, a molar absorption coefficient, and a quantum yield.

15-20. (canceled)

21. The fluorophore composition of claim 13, wherein the storage includes two or more freeze-thaw cycles.

22. A method of making a fluorophore composition, comprising combining an amount of a dye preparation with a hyaluronic acid salt solution, wherein the dye preparation comprises one or more types of fluorophore or fluorophore precursor.

23-27. (canceled)

28. The method of claim 22, wherein the amount of the dye preparation includes about 1 μg to about 10 μg of fluorophore or fluorophore precursor per milliliter of the fluorophore composition.

29. The method of claim 22 wherein the dye preparation comprises at least one of calcein AM, Hoechst 33342, Hoechst 33258, propidium iodide, or DAPI.

30. (canceled)

31. The method of claim 22, wherein the hyaluronic acid salt solution includes an amount of hyaluronic acid salt that is about 0.5 wt % to about 3.0 wt % of the fluorophore composition.

32. The method of claim 22, further comprising dividing the fluorophore composition into single-use aliquots.

33-36. (canceled)

37. A method of preserving a functionality of a fluorophore against degradation arising from storage, comprising: providing the fluorophore or a precursor thereof in a dye preparation; and combining an amount of the dye preparation with a hyaluronic acid salt solution to produce a fluorophore composition.

38. The method of claim 37, wherein the fluorophore or precursor is selected from calcein AM, Hoechst 33342, Hoechst 33258, propidium iodide, or DAPI.

39. The method of claim 37, wherein the fluorophore is selected from a nucleic acid dye and a cell viability dye.

40-43. (canceled)

44. The method of claim 37, wherein the functionality comprises one or more of a labeling efficiency, a molar absorption coefficient, and a quantum yield.

45-46. (canceled)

47. The method of claim 37, further comprising: dividing the fluorophore composition into single-use aliquots; and storing one or more of the single-use aliquots at a storage temperature of about −80° C. to about 4° C.

48-56. (canceled)
